(19) United States
(12) Patent Application Publication
(10) Pub. No.: US 2021/0260328 A1
(43) Pub. Date: Aug. 26, 2021

(54) SEAL WITH CURVED RIM

(71) Applicant: Intuitive Surgical Operations, Inc., Sunnyvale, CA (US)

(72) Inventor: Jake A. Luckman, New Haven, CT (US)

(21) Appl. No.: 17/055,112

(22) PCT Filed: May 8, 2019
(86) PCT No.: PCT/US2019/031390

§ 371 Date: Nov. 12, 2020

Related U.S. Application Data
(60) Provisional application No. 62/671,851, filed on May 15, 2018.

(51) Int. Cl. A61M 16/08; A61M 16/04

(52) U.S. Cl. CPC A61M 16/0816; A61M 16/0875; A61M 16/04

(57) ABSTRACT

A medical device may include a seal portion having a first seal wall extending to a first lip and a second seal wall extending distally to a second lip that contacts the first lip, the first lip and second lip defining a slit, and a rim portion extending around the seal portion, the rim portion being curved or bent in a proximal direction.

### CLAIM OF PRIORITY

This application claims the benefit of priority to U.S. Patent Application No. 62/671,851, filed on May 15, 2018, which is incorporated by reference herein in its entirety.

### TECHNICAL FIELD

This document relates generally to medical devices, and more particularly, to devices and methods for creating a seal in a surgical system.

### BACKGROUND

A surgical system may include a seal through which a surgical instrument can be inserted. In a teleoperated surgical system, a surgical instrument may be at the end of an instrument shaft inserted into the body of a patient, and the surgical instrument may be controlled by a user control device. In some systems, the instrument may “follow” the movement of the user control device.

### SUMMARY

An example medical device (Example 1) may include a seal portion and a bent rim portion extending around the seal portion. The bent rim portion may be curved or bent in a proximal direction. The seal portion may include a first seal wall extending to a first lip and a second seal wall extending distally to a second lip that contacts the first lip. The first lip and second lip may define a slit.

In Example 2, the medical device may also include an upper part and a lower part. For example, the lower part may be a swivel connector, and the upper part may be a cap. The rim portion may be compressed between the upper part and the lower part, which may flatten the rim portion between the upper part and the lower part. Flattening the rim portion may bias the first lip against the second lip.

In Example 3, the medical device of Example 2 may further include a septum seal between the upper part and the rim portion. The septum seal may be pressed against the rim portion by the upper part.

In Example 4, the medical device of Example 2 or 3 may include a seal plug. The seal plug may include an anchor, a stopper, and a connector shaped in a retroflex curvature between the anchor and the stopper. In some examples, a cross section of the connector at the retroflex curvature may have a width in a plane of the retroflex curvature and a thickness perpendicular to the plane of the retroflex curvature, and the width of the connector at the retroflex curvature may be larger than the thickness of the connector at the retroflex curvature.

In Example 5, the medical device of any one or any combination of Examples 2, 3, or 4, may be arranged or constructed such that the septum seal seals against the instrument shaft when the instrument shaft is inserted through the septum seal, and the first lip seals against the second lip to maintain a pressure when the instrument shaft is not inserted through the slit, in some examples, the medical device may include the surgical instrument.

In Example 6, the medical device of any one or any combination of Examples 1-5 may include a rib extending from the rim portion toward the slit. The rib may, for example, transfer forces from the rim portion to structures around the slit to close the slit.

In Example, 7, the medical device of any one or any combination of Examples 1-6 may include a rim portion that is symmetrical around a rim axis that is parallel to the slit.

An example medical device (Example 8) may include a first seal means having a first side and a second side that meet to form a seal, and a rim means for biasing the first and second side together when flattened. The seal means may, for example, be a single-slit seal, a tri-slit seal, a cross-slit seal, instrument seal, or another type of seal that has parts that may be biased together to form a seal.

In Example 9, the medical device of Example 8 may include a compression means for flattening the rim means. The compression means may, for example, include an upper part and a lower part that may be biased together (e.g., screwed, clamped, latched, pressed, or assembled) to compress the rim means.

In Example 10, the medical device of Example 9 may include a second seal means, such as a septum seal, or another slit seal, instrument seal, or other type of seal.

In Example 11, the medical device of Example 10 may be configured such that the compression means may compress the second seal means against the first seal means.

In Example 12, the medical device of Example 11 may be configured such that the second seal means seals against an instrument inserted through the second seal means and the first seal means, and the first seal means maintains a positive-end expiratory pressure when the instrument is not inserted through the first seal means.

In Example 13, the medical device of Example 12 may include a connector means for supplying ventilator gas to provide the positive-end expiratory pressure and securing means to secure the first seal means and second seal means to the connector means.

In Example 14, the medical device of Example 12 or 13 may include a surgical means that is insertable through the first seal means, wherein the first seal means maintains a pressure when the surgical means is not inserted through the first seal means.

In Example 15, the medical device of Example 14 may further include a teleoperated control means configured to operatively couple to the surgical means to allow a user to control the surgical means.

An example method (Example 16) of maintaining a positive-end expiratory pressure (PEEP) may include biasing opposed lips of a slit seal closed by flattening a bent or curved rim portion of the slit seal between an upper part and lower part of a medical device, wherein compressing the bent or curved portion of the slit seal biases the opposed lips of the slit seal against each other.

In Example 17, the method of Example 16 may include changing the shape of the rim portion of the slit seal by compressing the bent or curved rim portion of the slit seal.

In Example 18, the method of Example 17 may include flattening the rim portion.

In Example 19, the method of Example 16, 17, or 18 may include pressing a rim of a septum seal against the rim of the slit seal, wherein the rim of the septum seal compresses the rim of the slit seal.

Each of these non-limiting examples can stand on its own, or can be combined in various permutations or combinations with one or more of the other examples.

This Summary is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A is an illustration of clinical environment in which a seal may be used during a medical procedure.

FIG. 1B is an illustration of a manipulating system with which the example seal may be used.

FIG. 2A is a perspective view of the example seal shown in FIG. 2A.

FIG. 2B is a side view of an example seal.

FIG. 2C is a bottom perspective view of the example seal shown in FIG. 2A.

FIG. 2D is a top view of an example seal.

FIG. 2E is an illustration of an example seal with a rib.

FIG. 2F is a side view of an example seal with ribs.

FIG. 3 is a graph that illustrates pressure during phases of a respiratory cycle during a clinical procedure.

FIG. 4A is an exploded view of a medical device that includes the example seal shown in FIGS. 2A-2F.

FIG. 4B is a perspective view of the device shown in FIG. 4A.

FIG. 4C is a perspective view of the device shown in FIGS. 4A-4B with a plug inserted in a septum seal portion of the device.

FIG. 4D is a cross-sectional view of the device shown in FIGS. 4A-4C.

FIG. 4E is a cross-sectional view of the device shown in FIGS. 4A-4C with an endotracheal tube coupled to the device and a catheter shaft inserted into the device and endotracheal tube.

FIG. 5 is a flowchart illustration of an example method.

### DETAILED DESCRIPTION

### Overview

During a surgical procedure, it may be important to maintain a pressure in a body. For example, during a procedure in the lungs in which a patient may be on a ventilator (e.g., a biopsy), it may be clinically important to maintain a positive-end expiratory pressure. Maintenance of a positive-end expiratory pressure may, for example, provide a low pressure to partially inflate one or bath lungs, which may improve oxygen exchange in a patient and may be particularly important in a critically-ill patient. If a system leaks, the pressure may drop, which may compromise oxygen exchange in the lungs.

It may also be desirable to maintain an insufflation pressure during a laparoscopic procedure. For example, an incision may be made in the abdomen, and a cannula may be attached at the incision. The cannula may include a seal that may maintain an insufflation pressure and allow for passage of in instrument shaft through the seal and into an abdominal cavity, which may be inflated by the insufflation pressure, for example to create space for manipulating a tool such as an end effector or camera during a procedure.

To avoid or reduce leaks during a procedure, a seal may be designed to bias two sealing parts of the seal together. For example, a portion of a seal may be deformed from a natural state (e.g., flattened) to impart a bias or augment a natural bias.

In an example, a medical device seal may include a seal portion and a rim portion extending around the seal portion. The seal may have a first state and a second state. In the first state the seal is not assembled between a first part and a second part of a medical device and the rim portion has a first shape. In the second state the seal is assembled between the first part and the second part and the rim portion of the seal is biased into a second shape that is different than the first shape, and the seal portion is biased closed. In some examples, in the second state the rim portion is flattened, and in the first state the rim portion is not flattened. In some examples, in the first state the rim portion is curved.

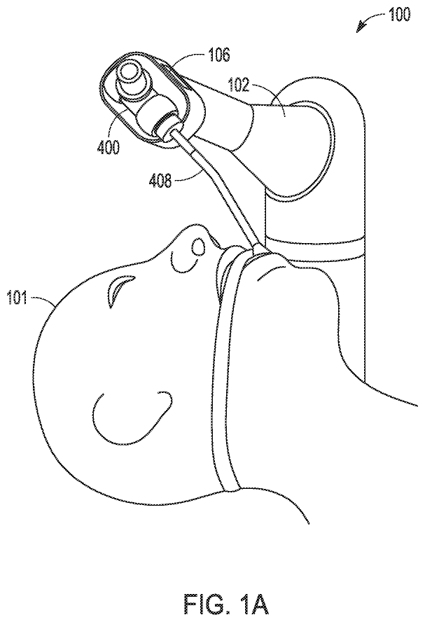

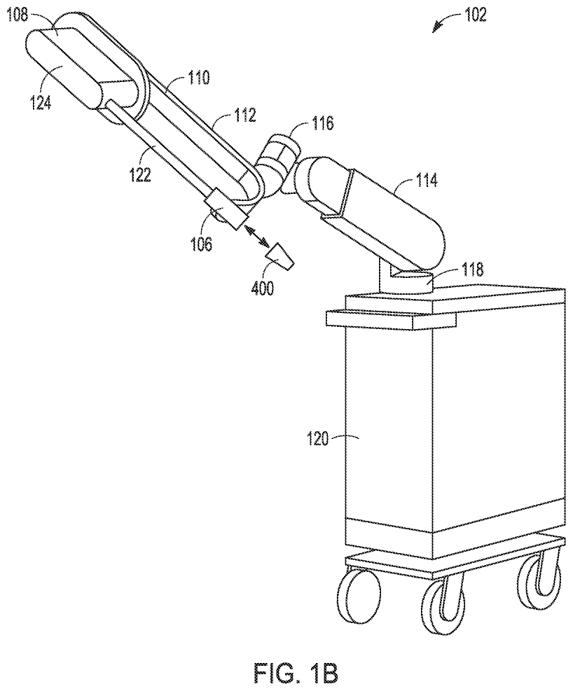

FIG. 1A is an illustration of clinical environment in which a patient 101 is situated on a surgical table with an endotracheal tube 408 inserted through the patient 101's mouth. The endotracheal tube 408 may be coupled to a medical device 400 (e.g., swivel connector), which may be coupled to a pressurized gas source (e.g., ventilator, not shown) that may provide a gas through the medical device 400 and into the endotracheal tube 408 and into the patient 101, e.g. to the patient 101's lungs. An elongated medical device 122 (e.g., instrument shaft or catheter) may be inserted through the medical device 400 and endotracheal tube 408 and into the patient 101. The elongated medical device 122 may be coupled to a manipulating system 102, such as the system shown in FIG. 1B, The manipulating system 102 may include an instrument mount 106 that may be coupled to a spar 108, which may be coupled to a manipulating arm 110, which may include a plurality of links 112, 114 that may be coupled by one or more joints 116, 118. The arm 110 may be mounted on a base 120, or alternatively may be mounted on a surgical table, ceiling, wall, or floor. The surgical instrument may be coupled to an instrument carriage 124, which may translate on the spar 108, and may optionally also rotate the instrument in response to user control inputs. The system may be operatively coupled to a user control system (not shown), which may be used to adjust the position of instrument mount 106 or to advance or retract the elongated medical device 122. For example, an instrument shaft may be inserted through the medical device 400 and endotracheal tube 408 and into the patient 101's lung, which may allow for performance of a biopsy procedure or other procedure.

Performance of a clinical procedure in the lungs may require ventilation of a patient 101, which may involve use of a seal through which an instrument shaft may pass, and which may prevent or reduce leakage of ventilator gas.

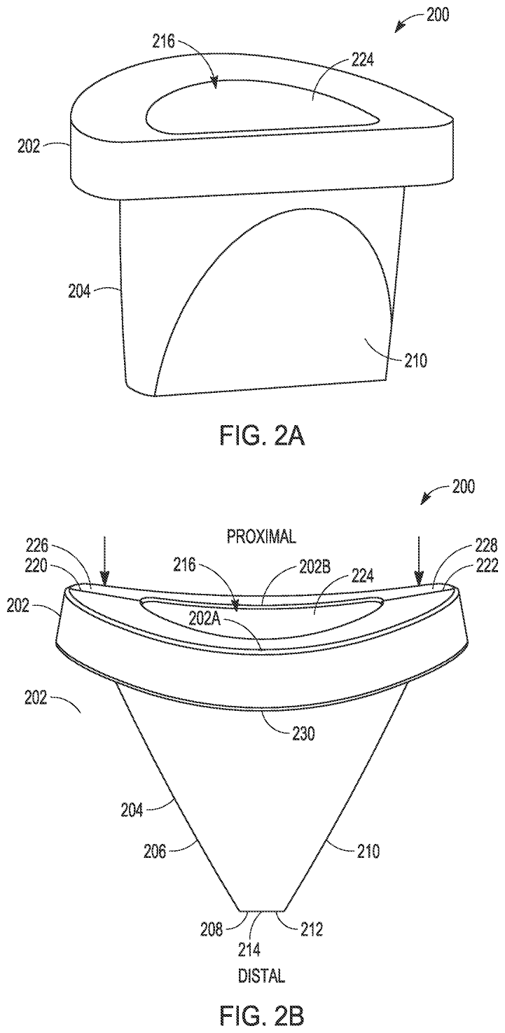

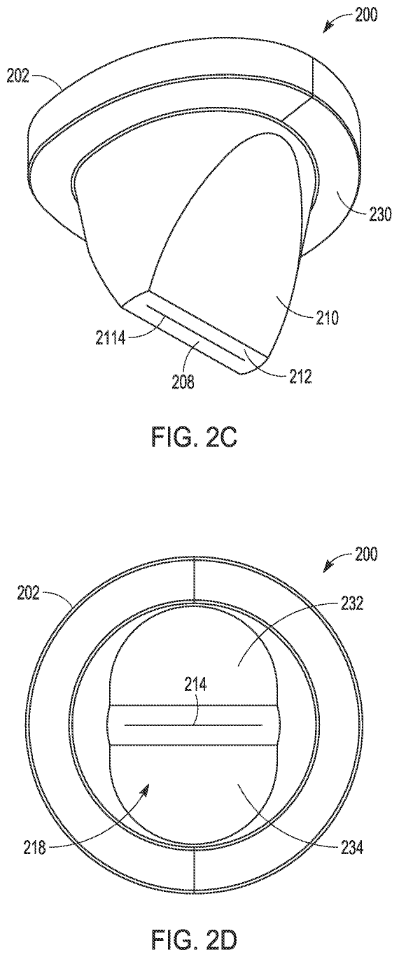

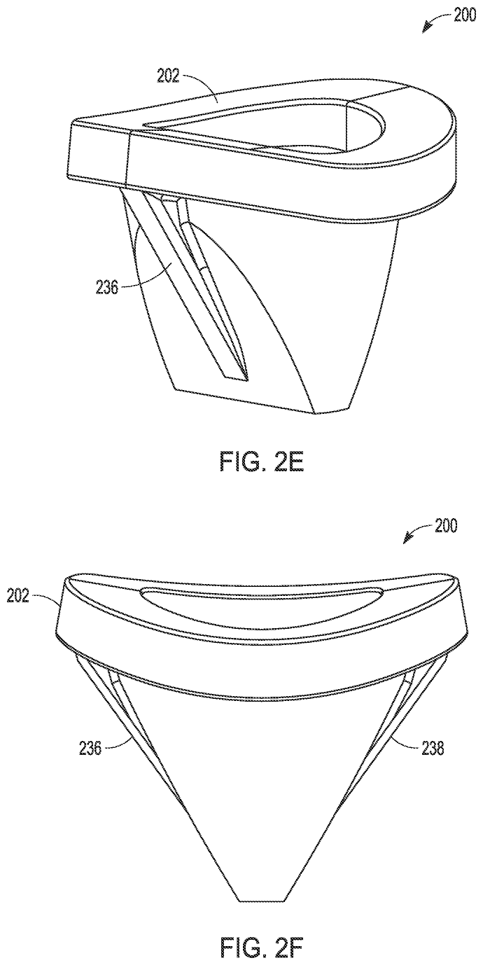

FIG. 2A-2F show an example seal 200. The seal 200 may, for example, be configured as a slit seal, popularly known as a “duckbill” seal. For clarity of explanation, with seal 200 will be referred to as a “slit seal” to avoid confusion with other seal components (e.g. septum seal 420). While a single slit is shown, in other examples, the seal 200 may be configured as such as a tri-slit seal or a cross-slit seal, or as another type of seal that has parts that may be biased together.

The seal 200 may include a rim portion 202 and a seal portion 204. The rim may have a consistent proximal-to-distal thickness (T). The seal portion 204 may also include a first side 206 that extends to a first lip 208, and a second side 210 that extends to a second lip 212. The first lip 208 and second lip 212 may meet to form a slit 214, shown in FIG. 2C and FIG. 2D.

The seal 200 may have portions that form a proximal opening 216. For example, one or more inner walls 224 of the rim portion 202 may define the opening 216. The rim portion 202 may extend partially or fully around the opening 216. An interior chamber 218 defined, at least in part, by interior surfaces of the seal portion 204 may extend distally from the opening 216 to the slit 214.

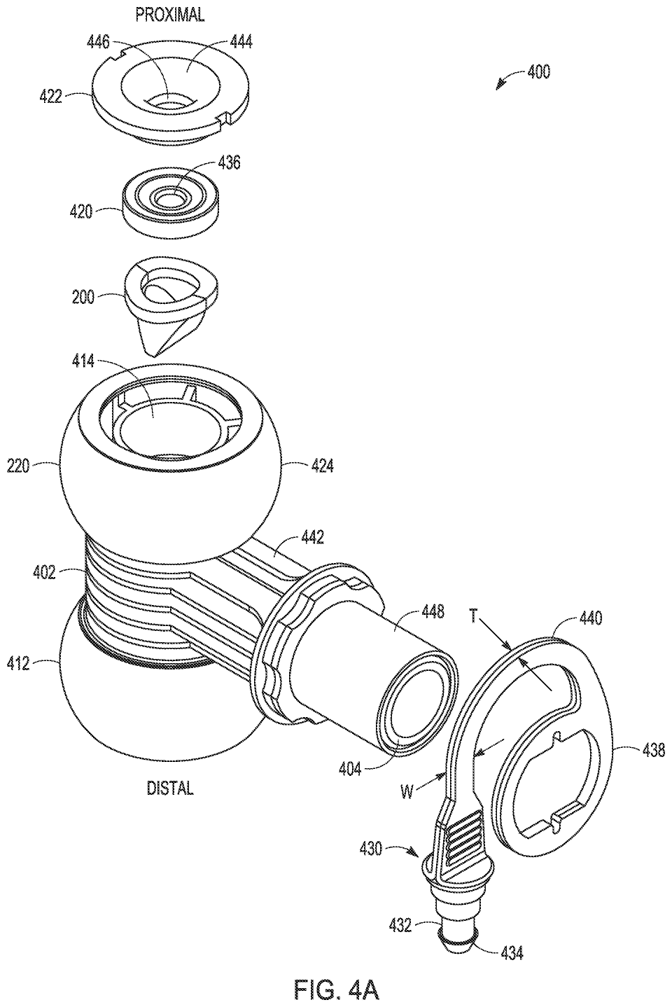

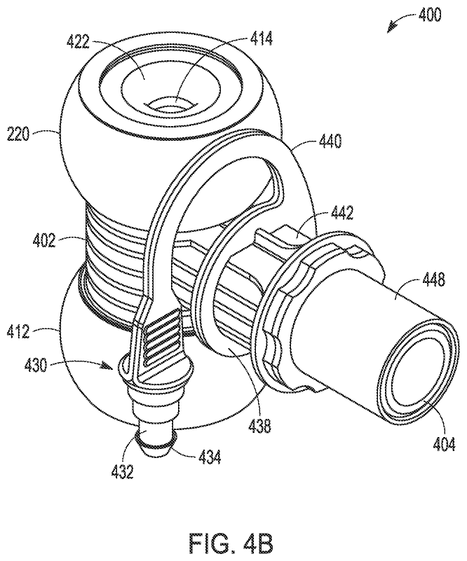

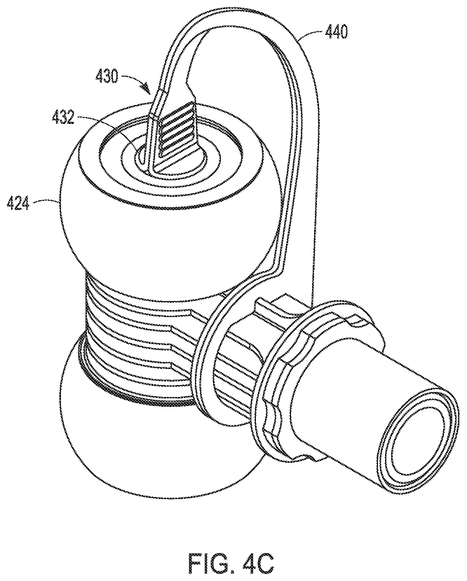

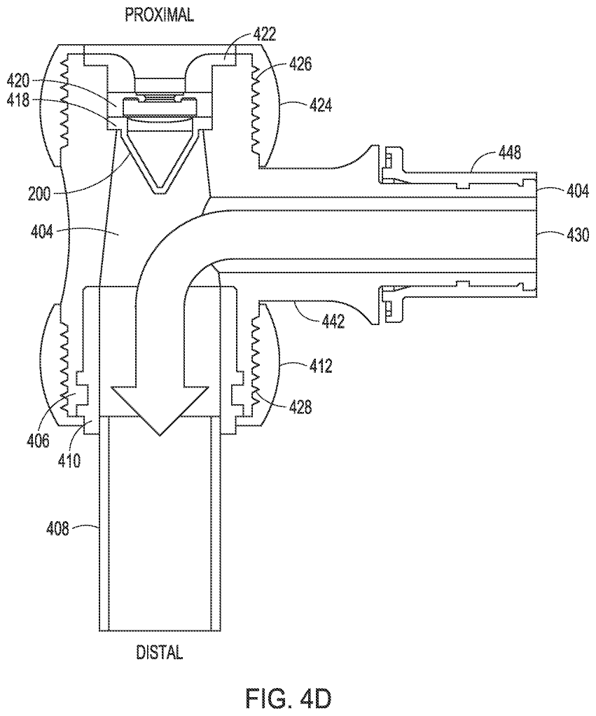

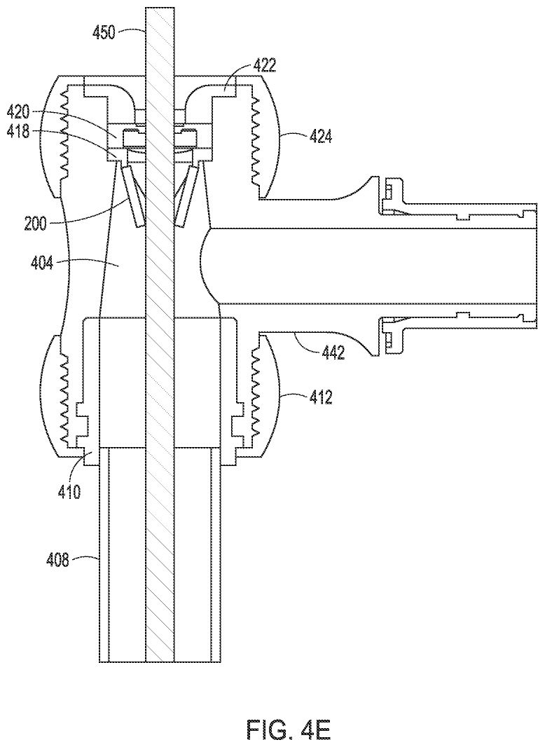

During a clinical procedure, an object such as an instrument and connected instrument shaft 450 (shown in FIG. 4D-4E) may be received through the opening 216 and pushed through the slit 214.

The rim portion 202 of the seal 200 may be bent or curved. For example, a first portion 220 and second portion 222 of the rim may extend upward (e.g., proximally). In various examples, the rim may be formed with a simple curve, or a complex curve, or two segments connected at an angular or rounded intersection, or a plurality of flat segments (e.g. a portion of an octagon or other polygon). The seal 200 may be sized, shaped, and configured so that a distal force (or distal component of a force) on the rim portion 202 imparts a bias on the first side 206 or second side 210 (or both), which in turn creates a bias on the first lip 208 and second lip 212 (or augments a natural bias) to close the slit 214.

The arrows in FIG. 2B show example force vectors on respective first and second top surfaces 226, 228 of the first portion 220 and second portion 222 of the rim portion 202. When a bottom surface 230 of the rim is pressed against another object (e.g., a lip 208, 212 on the connector body 402 as shown in FIG. 4D), the distally-directed forces may bias the first portion 220 and second portion 222 of the rim portion 202 downward, which pushes the respective first and second sides 206, 210 of the seal portion 204 downward to create the bias to close the slit 214. In some examples, the rim portion 202 may be flattened by the distally-directed forces, and the flattening of the rim may convey threes distally through the seal 200 to bias the sides of the seal 200 together. As shown in FIGS. 2E and 2F, in some examples, the seal 200 may optionally include one or more support structures 236, 238 (e.g., a rib), which may be sized and shaped to transfer a force from the rim portion 202 to press the lips 208, 212 toward a closed position.

In various examples, the seal 200 may be made of a biocompatible material, such as silicone rubber, urethane rubber, or polyisoprene. The seal 200 may be formed of a material with a durometer between 40 and 70 Shore A. To promote force transfer from the rim to the lips 208, 212 the seal 200 may be made of a material with a durometer between 60 and 70 Shore A. In some circumstances, a softer seal (e.g., 40-50 Shore A) may be desired. Support structures 236, 238 (e.g., ribs) may be particularly helpful when the seal 200 is made of a low-durometer material (e.g., a material with a Shore A 40-50 Shore A.)

Biasing the slit 214 closed may assist in establishing or maintaining a pressure during a clinical procedure. For example, the seal 200 may be used to establish or maintain an insufflation pressure during a laparoscopic procedure in the abdomen or a pressure in the lungs. The seal 200 may be used to maintain a positive-end expiratory pressure (“PEEP”), e.g. to maintain a positive pressure during an end expiratory phase of a respiration cycle.

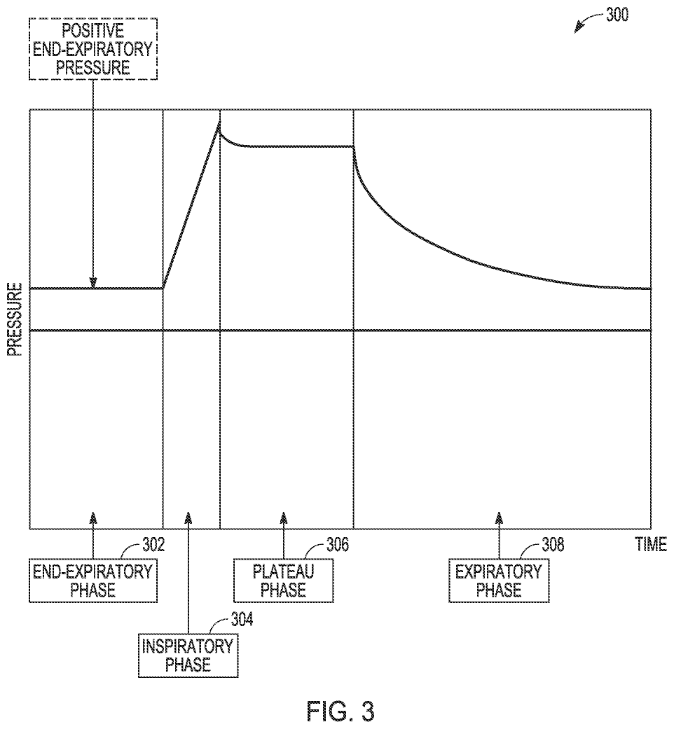

FIG. 3 shows a pressure curve 300 in an example respiratory cycle. A pressure may be provided by a pressure source in a system, such as the system 100 shown in FIG. 1A. The phases of the respiration cycle are labeled in the figure. The X-axis in the graph represents time during the respiration cycle, and the Y-axis represents pressure (e.g., cm H₂O or mm Hg.) The pressures value may represent the pressure in a patient 101's lungs, or in a system 100 providing pressures to the patient 101's lungs, or both in a system 100 where pressure is mostly consistent through the system 100. In an inspiratory phase 304, the pressure rises as the system 100 pushes air (or oxygen or an appropriate mixture of gasses) into the patient 101's lungs. During a plateau phase 306, the pressure may drop from the end of the inspiratory phase 304, and then level off at a steady level, which may be controlled by a system 100 according to a specified plateau phase 306 pressure. During an expiratory phase 308, the pressure drops off as the system 100 lowers the applied pressure to allow air to exit the lungs. The end expiratory phase 302 occurs after the expiratory phase 308 (e.g., the right side of the graph connects to the left side of the graph as the cycle starts over.) The end expiratory phase 302 may considered as an extension of the expiratory phase 308, i.e. the end of the expiratory phase 308, in which the pressure in the expiratory phase 308 has dropped to a dropped to a steady state value (or range) or to a lower threshold (e.g., if there is a leak in the system 100 the pressure may continue to drop during the end expiratory phase 302 as air leaks out of the system 100).

If a leak is present in the system 100, the pressure in the patient 101's lungs may drop more than desired during one or more of the phases in the respiratory cycle. A pressure drop from a leak may be especially relevant or impactful during the end expiratory phase 302, in which the system 100 may not provide much air flow that could compensate for a leak. The seal structure shown in FIGS. 2A-2F may help avoid the occurrence or size of such leaks and maintain a positive end expiratory pressure.

FIGS. 4A-4E show an example medical device 400 that may include the seal 200 shown in FIGS. 2A-2F. The medical device 400 may be a connector, such as a swivel connector.

The device 400 may include a body 402 that may include portions defining an internal cavity 414, which may receive one or more seal components (described below), The body 402 may include a port 448, which may facilitate coupling to a pressure source (not shown) such as a ventilator.

A distal portion 406 of the body 402 may be coupled to an endotracheal tube 408, which may be extended into a patient 101, such as into a portion of the respiratory system of the patient 101 (e.g., into the trachea or into the lungs.) In some examples, an adaptor 410 may be rotatably coupled to the body 402 and also coupled to the endotracheal tube 408 (shown in FIG. 1A), which may allow the body 402 to swivel with respect to the endotracheal tube 408. A lower end cap 412 may be coupled to the distal portion 406 of the body 402, for example using a threaded connection 428. The lower end cap 412 may couple the swivel part to the body 402. For example, the lower end cap 412 may retain the adaptor 410 inside a lower portion 416 of the internal cavity 414 of the body 402.

One or more sealing parts may be assembled into the body 402. For example, the seal 200 shown in FIGS. 2A-C may be assembled into the body 402. As shown in FIG. 4D, the bottom surface 230 of the rim portion 202 may be pressed against a lip 418 in the internal cavity 414. The seal 200 may be pressed against the lip 418, which may create or augment a bias to press the slit 214 closed.

In some examples, a septum seal 420 and cap 422 may also be assembled into the body 402. The septum seal 420 may be assembled on top of the seal 200, and the cap 422 may be assembled on top of the septum seal 420, as shown in FIG. 4D. The cap 422 may include a guide portion 444, which may be sized and shaped to guide an instrument toward an opening 446 in the cap 422.

The seal 200, septum seal 420, and cap 422 may be retained in the cavity 414 in the body 402 by a top end cap 424, which may, for example, be coupled to the body 402 by a threaded connection 426. The connection between the top end cap 424 and body 402 may provide compression forces to flatten the rim portion 202 of the seal 200 against the lip 418 on the body 402 to bias the lips 208, 212 of the seal 200 together to close the slit 214, A ventilator gas (e.g., air or an oxygen mixture) may be supplied through a passageway in the port 448 (as indicated by the arrow). The supplied ventilator gas may establish a pressure in the cavity 404 below the seal 200 and in the endotracheal tube 408. The pressure in the system 100 (connector, intubation tube, and optionally also inside a patient 101's lungs) may be controlled according to specified parameters, e.g., to create a pressure profile as shown in FIG. 3. The pressure in the cavity 404 may be maintained by the seal 200.

A plug 430 may provide an additional seal against leakage. The plug 430 may include a stopper 432 that may be sized and shaped to seal the septum seal 420, e.g., the stopper 432 may be sized and shaped to be inserted into orifice 436 in the septum seal 420 as shown in FIG. 4C. The stopper 432 may be any suitable shape to plug the septum seal 420. For example, the stopper 432 may include a body having a cross-section that matches the shape of the orifice 436 in the septum seal 420. The stopper 432 may have a lip 434 (shown in FIG. 4A), which may retain the plug 430 in the septum seal 420 when the lip 434 is pushed through the orifice 436 in the septum seal 420.

The plug 430 may also include an anchor 438 and a connector 440 that couples the anchor to the stopper 432. The anchor 438 may be sized and shaped to anchor the plug 430 to the body 402 of the connector 440. The anchor 438 may be any suitable shape to anchor the plug 430 to the body 402. For example, the body 402 may include (or may be coupled to) a neck portion 442, and the anchor 438 may be coupled to or extend around the neck portion 442.

The connector 440 may be shaped in a retroflex curvature between the anchor 438 and the stopper 432. A cross section of the connector 440 at the retroflex curvature may have a width (w) in a plane of the retroflex curvature that is larger than a thickness (t) perpendicular to the plane of the retroflex curvature.

FIG. 4E shows an instrument shaft 450 inserted through the septum seal 420 and through the seal 200. The septum seal 420 may seal against instrument shaft 450 to avoid leakage of gas out through the proximal side of medical device 400 (e.g., past the slit seal 200 and out of the body 402.) The sides of the seal 200 may separate to open the slit 214 to allow passage of the instrument shaft 450. In various examples, the instrument shaft 450 may be a portion of catheter (which may optionally provide a conduit for other instruments, or may be a camera shaft or shaft for another instrument.

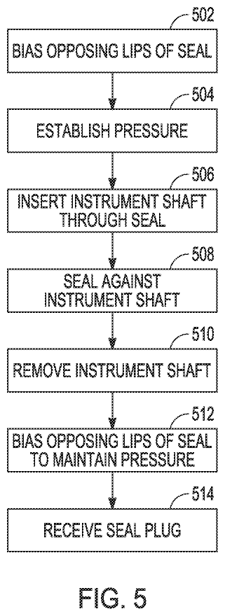

FIG. 5 is a flowchart illustration of an example method 500. At 502, opposing lips 208, 212 of a slit seal 200 may be biased together. For example, a curved or bent rim of a slit seal 200 may be flattened to bias the lips 208, 212 together. At 504, a pressure may be established, e.g., using a ventilator. The pressure may be cyclical, e.g., to establish a respiration cycle, as illustrated in FIG. 3. At 506, an instrument shaft 450 may be inserted through the seal 200. At 508, the instrument shaft 450 may, for example, seal against a septum seal 420 and extend through the slit seal 200, such that the septum seal 420 prevents leakage of ventilator gas even though the slit seal 200 may be compromised (e.g., the slit seal 200 may not seal against the shaft but the septum seal 420 prevents leakage.) At 510 the instrument shaft 450 may be removed. At 512, the opposing lips 208, 212 of the slit seal 200 are biased together to re-form the seal 200 at the slit 214 and maintain a pressure (e.g., a positive post end-expiratory pressure.) For example, forces in a flattened rim of a seal 200 may bias the opposing lips 208, 212 together (or augment a natural bias) to close the slit seal 200. At 514, a seal plug 430 may optionally be inserted into the septum seal 420 to provide a secondary seal, e.g., to further avoid leakage of ventilator gas.

In various examples, a user control system may be used to move an instrument carriage 124, catheter, instrument shaft 450, or instrument (e.g., end effector or camera or to control a camera, end effector, or other tool that is inserted through a slit seal 200 or medical device 400 as described above. In various examples, multiple instrument shafts 450 and respective instruments (e.g., a camera and an end effector) may be coupled to an arm 110, or a system 100 may include a plurality of arms 110, each of which may be coupled to an instrument shaft 450 or a single instrument shaft 450 may include both a camera and an end effector. A plurality of seals 200 may be used (e.g., one seal 200 per shaft or per entry point to the patient 101.) While the examples have been explained in-depth with respect to an endotracheal procedure, the example seals 200, devices, systems, and methods described herein may be applied in other types of procedures, such as a laparoscopic procedure in the abdomen or elsewhere.

Persons of skill in the art will understand that any of the features described above may be combined with any of the other example features, as long as the features are not mutually exclusive. All possible combinations of features are contemplated, depending on clinical or other design requirements. In addition, if manipulating system units are combined into a single system 100 (e.g., telesurgery system), each individual unit may have the same configuration of features, or, one patient-side unit may have one configuration of features and another patient-side unit may have a second, different configuration of features.

The examples (e.g., methods, systems, or devices) described herein may be applicable to surgical procedures, non-surgical medical procedures, diagnostic procedures, cosmetic procedures, and non-medical procedures or applications. The examples may also be applicable for training, or for obtaining information, such as imaging procedures. The examples may be applicable to handling of tissue that has been removed from human or animal anatomies and will not be returned to a human or animal, or for use with human or animal cadavers. The examples may be used for industrial applications, general robotic uses, manipulation of non-tissue work pieces, as part of an artificial intelligence system, or in a transportation system.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof) with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system 100, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round”, a component that is not precisely circular (e.g., one that is slightly oblong or is a many-sided polygon) is still encompassed by this description. Coordinate systems or reference frames are provided for aiding explanation, and implementations may use other reference frames or coordinate systems other than those described herein.

The above description is intended to be illustrative, and not restrictive. For example, the above-described examples (or one or more aspects thereof) may be used in combination with each other. Other embodiments may be used, such as by one of ordinary skill in the art, upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments may be combined with each other in various combinations or permutations. The scope of the invention should be determined with reference to the appended claims, along with the full scope of equivalents to which such claims are entitled.

1. A medical device comprising:
a seal portion including a proximal opening, a distal slit defined by a first lip and a second lip, a first seal wall extending from the proximal opening distally to the first lip, and a second seal wall extending from the proximal opening distally to the second lip; and
a flexible rim portion extending around the proximal opening of the seal portion,
wherein the rim portion in an uncompressed state is curved in a proximal direction.

2. The medical device of claim 1, wherein:
the medical device further comprises an upper part and a lower part; and
the rim portion in a compressed state between the upper part and the lower part.

3. The medical device of claim 2, wherein:
in the compressed state the rim portion biases the first lip against the second lip to close the slit.

4. The medical device of claim 2, wherein the lower part is a swivel connector and the upper part is a cap.

5. The medical device of claim 2, wherein:
the medical device further comprises a septum seal between the upper part and the rim portion; and
the septum seal is pressed against the rim portion by the upper part.

6. The medical device of claim 5, wherein:
the medical device further comprises a seal plug including an anchor, a stopper, and a connector shaped in a retroflex curvature between the anchor and the stopper;
a cross section of the connector at the retroflex curvature including a width in a plane of the retroflex curvature and a thickness perpendicular to the plane of the retroflex curvature; and
the width of the connector at the retroflex curvature is larger than the thickness of the connector at the retroflex curvature.

7. The medical device of claim 5, wherein:
the septum seal seals against an instrument shaft when the instrument shaft is inserted through the septum seal; and
the first lip seals against the second lip to maintain a pressure distal of the seal portion when the instrument shaft is not inserted through the slit.

8. The medical device of claim 1, wherein:
the medical device further comprises a rib extending from the rim portion toward the slit.

9. The medical device of claim 1, wherein:
the rim portion is symmetrical around a rim axis that that is parallel to the slit.

10. A medical device comprising:
means for forming a seal; and
means for biasing the seal closed by moving from a curved state to a flattened state;
wherein the means for biasing is coupled to the means for forming opposite the seal.

11. The medical device of claim 10, wherein:
the medical device further comprises means for flattening the means for biasing.

12. The medical device of claim 11, wherein:
the medical device further comprises means for forming a second seal; and
the means for flattening comprises means for compressing the means for forming a second seal against the for forming a seal.

13. The medical device of claim 12, wherein:
the means for forming a seal comprises means for maintaining a positive-end expiratory pressure when means for performing a medical procedure is not inserted through the means for forming a seal; and
the means for forming a second seal comprises means for maintaining, the positive-end expiratory pressure when the means for performing a medical procedure is inserted through the means for forming a seal.

14. The medical device of claim 13, wherein:
the medical device further comprises means for providing the positive-end expiratory pressure and means for securing the means for providing to the means for forming a seal.

15. The medical device of claim 11, wherein:
the medical device further comprises means for performing a medical procedure inserted through the means for forming a seal; and
the means for forming a seal comprises means for maintaining a pressure distal of the means for forming a seal when the means for performing a medical procedure is not inserted through the means for forming a seal.

16. The medical device of claim 15, wherein:
further comprising a teleoperated control means configured to operatively couple to the surgical means to allow a user to control the surgical means; and
the medical device further comprises means for teleoperation control of the means for performing a medical procedure.

17. A method of maintaining a positive-end expiratory pressure (PEEP) during a medical procedure, the method comprising:
biasing opposed lips of a slit seal closed by flattening curved rim portion of the slit seal.

18-19. (canceled)

20. The method of claim 117, wherein:
flattening the curved rim portion of the slit seal includes pressing a rim of a septum seal against the curved rim portion of the slit seal.

21. The medical device of claim 1, wherein:
the rim portion is saddle shaped.

22. The medical device of claim 1, wherein:
the rim portion includes a first portion that curves proximally and a second portion that curves distally.

23. The medical device of claim 1, wherein:
the rim portion includes a pair of peaks and a pair of valleys;
the pair of peaks are located on opposing sides of the rim portion and curve proximally; and
the pair of valleys are located on opposing sides of the rim portion and curve distally.

24. A medical device seal comprising:
a saddle-shaped rim including a first portion that curves proximally and a second portion that curves distally; and
a seal portion connected to the saddle-shaped rim and including:
a first lip;
a second lip opposite the first lip;
a slit seal defined by the first and second lips;
a first seal wall extending distally from the saddle-shaped rim to the first lip; and
a second seal wall extending distally from the saddle-shaped rim to a second lip.
